(19) United States
(12) Patent Application Publication
(10) Pub. No.: US 2005/0010096 A1
(43) Pub. Date: Jan. 13, 2005

(54) EKG enabled apparel with detachable electronics

(76) Inventor: Thomas P. Blackadar, Natick, MA (US)

Correspondence Address: Robert M. Abrahamsen;Wolf, Greenfield & Sacks, P.C., 600 Atlantic Avenue, Boston, MA, 02210, US

(21) Appl. No.: 10/844,965

(22) Filed: May 13, 2004

Related U.S. Application Data
(60) Provisional application No. 60/470,060, filed on May 13, 2003.

(51) Int. Cl. A61B 5/04

(52) U.S. Cl. 600/388; 600/393

(57) ABSTRACT

A garment such as a shirt or sports bra has first electrodes attached thereto so as to sense at least one electronic signal from a person wearing the garment. An electronic enclosure, which may include second electrodes that are configured and arranged to electronically interface with the first electrodes, may be releasably attached to the garment. The electronic enclosure may, for example, comprise an EKG sensor.

This application claims the benefit of Provisional Application Ser. No. 60/470,060, filed May 13, 2003, which is incorporated herein by reference in its entirety.

### BACKGROUND

The current state of art in EKG monitoring uses anywhere from 2 leads to 12 leads to capture the signal. There are many two lead EKG products available on the market, however they either go across the hands as in bicycle grips, or across the chest as in the chest straps made by Polar.

### SUMMARY

According to one aspect of the invention, a garment such as a shirt or sports bra has first electrodes attached thereto so as to sense at least one electronic signal from a person wearing the garment. An electronic enclosure, which may include second electrodes that are configured and arranged to electronically interface with the first electrodes, may be releasably attached to the garment. The electronic enclosure may, for example, comprise an EKG sensor.

### BRIEF DESCRIPTION OF THE DRAWINGS

FIGS. 1a-d show an example embodiment of a system embodying various aspects of the present invention;

FIG. 2 illustrates an example embodiment of an additional feature that may be employed to help secure an EKG sensor to a garment;

FIGS. 3-5 illustrate a variety of examples of how garments may alternatively be configured in accordance with various aspects of the invention;

FIG. 6 shows an example embodiment of an electronic enclosure embodying various aspects of the present invention; and

FIG. 7 illustrates another example embodiment of an additional feature that may be employed to help secure an EKG sensor to a garment.

### DESCRIPTION

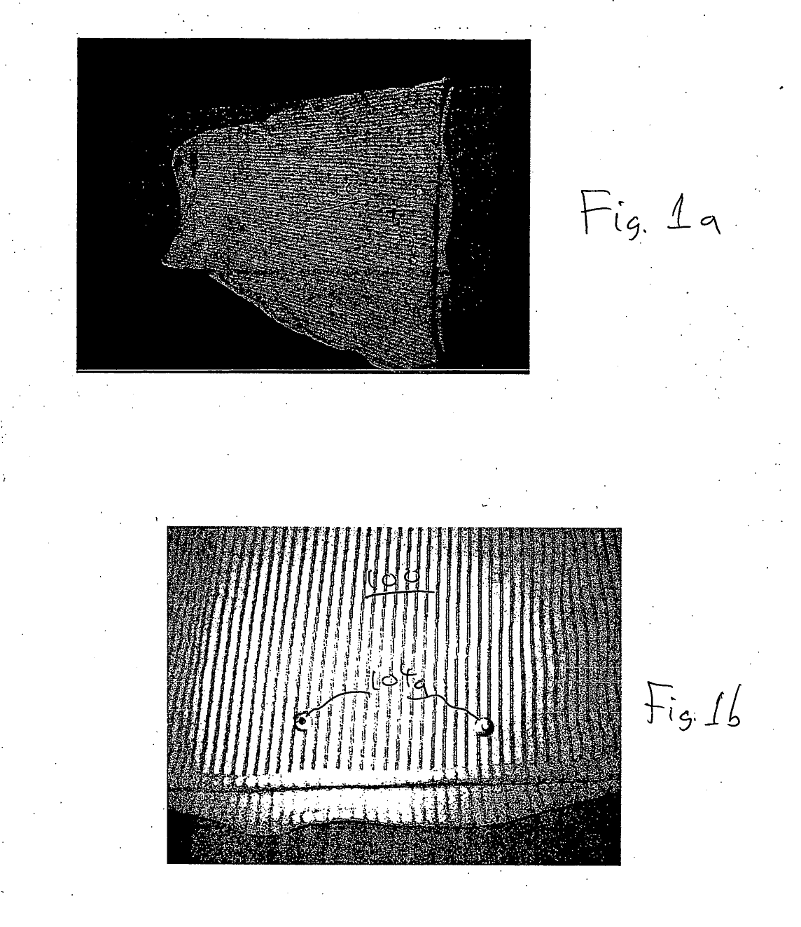

FIGS. 1a-d show an example embodiment of a system embodying various aspects of the present invention. As shown, the system includes a garment 100 (a shirt in the illustrative example shown) to which a plurality of EKG pickup contacts 102 (e.g., carbonized rubber electrodes, or metal rivets, snaps or buttons) are fixedly attached so that they can detect an EKG signal from the body of the person wearing the garment 100. Also attached to the garment 100 are a pair of EKG conductive snaps 104a that are adapted to releasably mate with a corresponding pair of conductive snaps 104b fixedly attached to the housing of an EKG sensor 106. The contacts 102 may be electrically connected to the snaps 104a via conductive threads sewn into the shirt. Preferably, the conductive threads are sewn in a zigzag pattern so as to provide the elasticity required to keep the threads from breaking and from becoming uncomfortable. In the example shown, one half (i.e., four) of the contacts 102 are connected to one of the snaps 104b, and the other half (i.e., the other four) of the contacts 102 are electrically connected to the other snap 104b. Thus, when the snaps 104b of the EKG sensor 106 are attached to the snaps 104a on the garment, the EKG sensor 106 can detect an EKG signal picked up by the contacts 102.

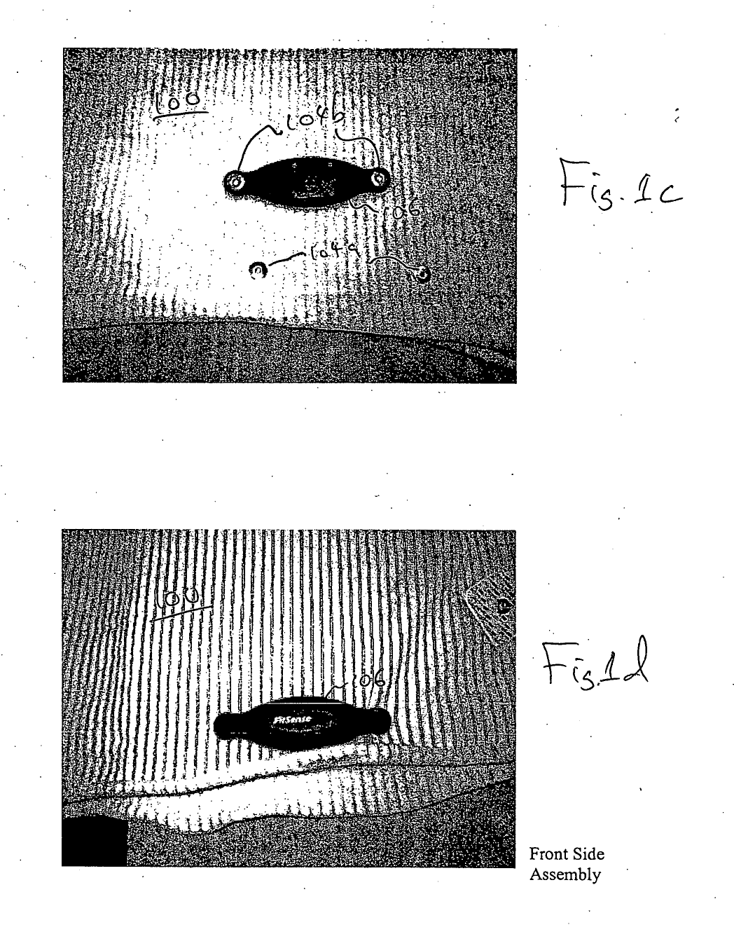

FIGS. 2 and 7 illustrate an example embodiment of an additional feature that may be employed to help secure the EKG sensor 106 to a garment. As shown, a section of a strap of material 100 may be sewn or otherwise attached to the garment 100, with one portion 202 of the strap 100 including the hook portion and another portion 204 including the loop portion of a hook and loop fastener. The straps 100 may then be wrapped around the EKG sensor 106, and the hook portion 202 may be fastened to the loop portion 204 to secure the EKG sensor firmly in place. Of course, other fastening techniques and mechanisms other than hook and loop fasteners may additionally or alternatively be employed.

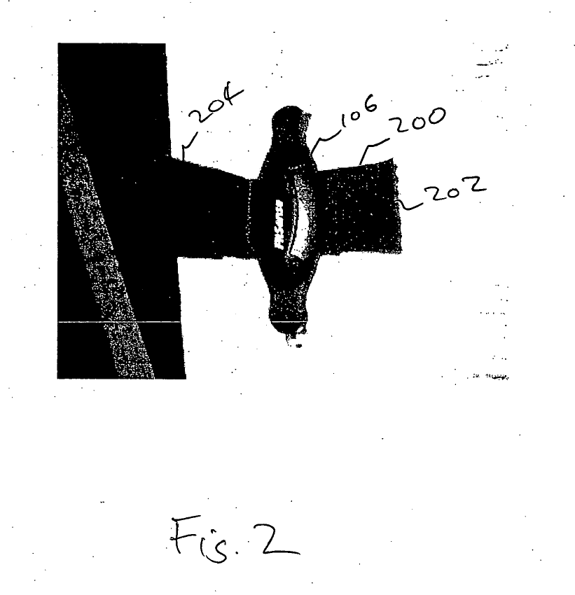

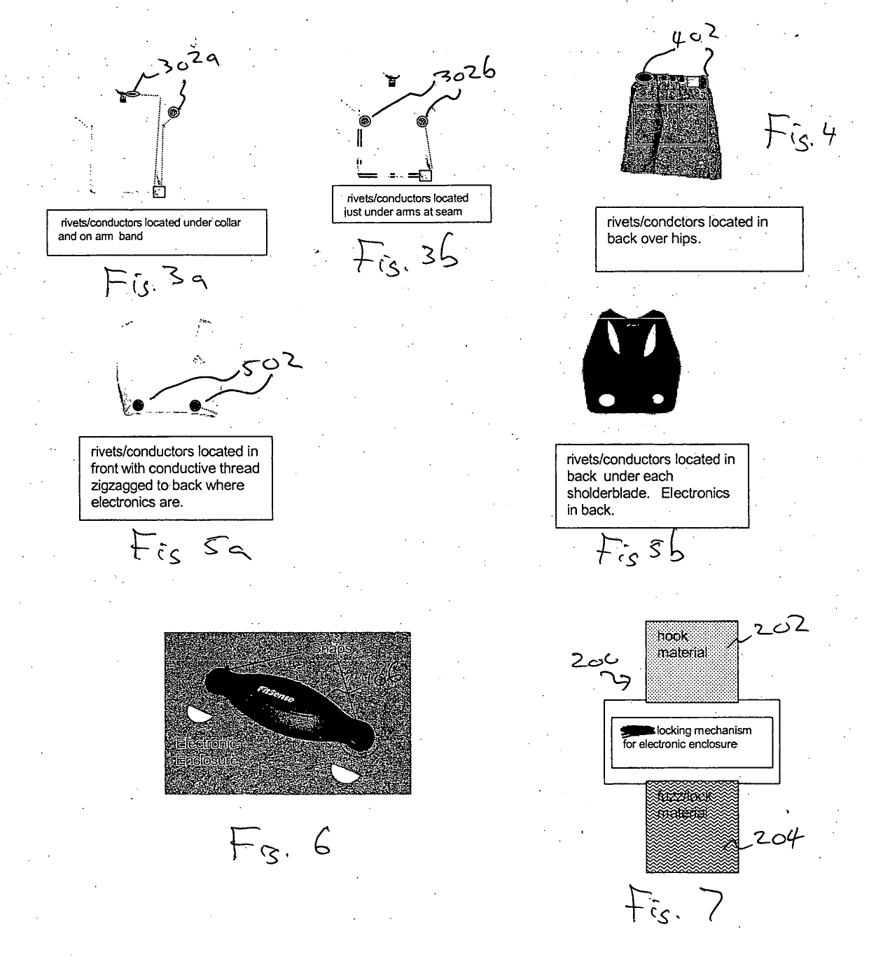

FIGS. 3-5 illustrate a variety of examples of how garments may alternatively be configured in accordance with various aspects of the invention. Each article of clothing has been modified in a fashion that maintains comfort and is not restrictive in any way. Unique ways of fastening the electronic assembly have been invented, unique methods of obtaining the electrical signals of the heart have been employed, and unique placements of electrodes have been uncovered that may enable comfortable placement within a garment. Each garment may use a detachable electronic enclosure 106 for the interpretation of the signal. The areas 302a, 302b, 402 and 502 represent examples of possible detection points of the EKG signal in the clothing. By locating electrodes properly, e.g., on the front and/or back of a bra; on an elastic collar, an elastic sleeve, and/or under the arms of a shirt; and/or on the rear elastic waistband of shorts or underwear, the EKG signal can be adequately detected to determine heart rate.

One embodiment of the design uses conductive threads in a zigzag flexible stitch. The zigzag stitch provides the elasticity required to keep the threads from breaking and from becoming uncomfortable. By providing one or more runs of conductive threads, proper impedance matching can be done for the circuit so as to limit the electrical losses.

As mentioned above, the electronic enclosure 106 may be an intelligent EKG sensor with a complete waveform processing system. This waveform processing system may provide, for example, an accurate beat-to-beat interval of the R-Wave of the heart rate waveform. Other processing may additionally or alternatively be performed on this wave form to detect arrhythmias and to set one or more alarm point(s). The beat-to-beat interval may be translated into heart rate in beats per minute. A wireless interconnect may be used to transmit the data to a receiving device where the heart rate can be stored, displayed or further processed with other physiological data. It should be appreciated that, in alternative embodiments, any other electronic device (i.e. other than an EKG sensor) that receives as an input an electronic signal picked up from contacts 102 may additionally or alternatively be housed within the enclosure 106.

The enclosure may use medical grade snaps in order to connect to the apparel. The snaps may be located on the apparel just outside the measured dimensions of the snap placement. By using stretch material, the snaps may pull the enclosure tight against the fabric.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The invention is limited only as defined by the following claims and the equivalents thereto.

1. A garment having first electrodes attached thereto so as to sense at least one electronic signal from a person wearing the garment.

2. The garment of claim 1, wherein the garment is a shirt.

3. The garment of claim 2, wherein the first electrodes are attached to at least one of a snug fitting collar and a snug fitting sleeve of the shirt.

4. The garment of 3, wherein the stretch material in the at least one of the snug fitting collar and the snug fitting sleeve is done with conductive thread in a zigzag stitch.

5. The garment of claim 3, wherein respective first electrodes are attached to each of the snug fitting collar and the snug fitting sleeve of the shirt.

6. The garment of claim 1, wherein the garment comprises pants or shorts.

7. The garment of claim 1, wherein the garment comprises a bra.

8. The garment of claim 1, wherein the bra is a sports bra.

9. The garment of claim 1, in combination with an electronic enclosure releasably attachable to the garment, the electronic enclosure including second electrodes that are configured and arranged to electronically interface with the first electrodes.

10. The garment of claim 1, further comprising second electrodes attached to the shirt, at least one pair of the second electrodes being electrically coupled to at least one pair of the first electrodes via at least one pair of conductive threads.

11. The garment of claim 10, wherein the conductive threads are sewn in the garment using a zigzag stitch.

12. The garment of claim 10, in combination with an electronic enclosure releasably attachable to the garment, the electronic enclosure including third electrodes that are configured and arranged to electronically interface with the second electrodes.

13. The combination of claim 12, wherein the third electrodes are configured and arranged to releasably attach to the second electrodes so that the electronic enclosure may be releasably attached to the garment.

14. The combination of claim 9, wherein the electronic enclosure comprises an EKG sensor.

15. The combination of claim 14, wherein the EKG sensor comprises an ECG front-end amplifier to amplify a heart-rate signal picked up by the first electrodes.

16. The combination of claim 15, wherein the EKG sensor is configured to process the amplified heat-rate signal to generate heart rate data.

17. The combination of claim 16, wherein the EKG sensor comprises a transmitter to transmit the processed heart rate data to a receiving device.

18. The combination of claim 16, wherein the EKG sensor is configured process the heart rate data to generate alarms, and further comprises a transmitter to transmit the alarms to a receiving device.

19. The combination of claim 10, wherein the distance between the second electrodes when the garment is in a relaxed state is substantially less than the distance between the third electrodes.

20. The combination of claim 19, wherein the distance between the second electrodes when the garment is in a relaxed state is approximately one half the distance between the third electrodes.
